(19) United States
(12) Patent Application Publication
(10) Pub. No.: US 2025/0069733 A1
(43) Pub. Date: Feb. 27, 2025

(54) System, Method, and Computer Program Product for Determining a Care Provider Based on a Care Request

(71) Applicant: Orchestrall Healthcare Limted, North Point (HK)

(72) Inventors: Man-Cheung Hau, Villanova, PA (US); Daniel Allen Hirschfeld, Tomonium, MD (US)

(21) Appl. No.: 18/947,855

(22) Filed: Nov. 14, 2024

Related U.S. Application Data
(63) Continuation of application No. 17/328,087, filed on May 24, 2021, now abandoned.
(60) Provisional application No. 63/028,829, filed on May 22, 2020.

(51) Int. Cl. G16H 40/20; G06Q 40/08

(52) U.S. Cl. CPC G16H 40/20; G06Q 40/08

(57) ABSTRACT

Provided are computer-implemented methods for determining a care provider based on a care request which may include receiving data associated with a care request, the data associated with the care request transmitted by a device associated with a care recipient; determining one or more values corresponding to one or more parameters of the care request; determining one or more weighted scores corresponding to one or more care providers based on the one or more values corresponding to the one or more parameters of the care request; and selecting a care provider from among the one or more care providers based on the one or more weighted scores. In some non-limiting embodiments or aspects, methods may include generating a care request for the care provider selected from among the one or more care providers. Systems and computer program products are also provided.

### CROSS REFERENCE TO RELATED APPLICATIONS

This application is a continuation-in-part of U.S. patent application Ser. No. 17/328,087 filed May 24, 2021, which claims the benefit of U.S. Provisional Application No. 63/028,829 filed May 22, 2020, both of which are incorporated herein by reference in their entireties.

### BACKGROUND

### Technical Field

This disclosure relates generally to providing response to care requests and, in some non-limiting embodiments or aspects, to systems, methods, and computer program products for determining a care provider based on a care request.

### Technical Considerations

Care providers (e.g., a clinician such as a doctor, a physical therapist, and/or the like, a relative, a friend, and/or the like) caring for a care recipient (e.g., a patient, a customer, and/or the like) may receive a request from the care recipient or family caregiver for services (e.g., a request to schedule an appointment). The individual caring for the care recipient may then determine when to schedule the request for services. However, the individual may not be able to accommodate the request from the care recipient for services as quickly and/or may not be as well suited to accommodate the request as another individual. For example, the individual may be trained to generally provide the services but may not be an individual who specializes in the services. Additionally, the identity of another individual may not be known to the individual caring for the care recipient. As a result, the care recipient may wait until the individual caring for the care recipient is available, resulting in a potential delay in the care recipient receiving services from an individual who is otherwise capable of providing the services requested.

### SUMMARY

Accordingly, disclosed are systems, methods, and computer program products for determining a care provider based on a care request.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

FIG. 1 is a diagram of a non-limiting aspect or embodiment of a system for determining a care provider based on a care request;

FIG. 2 is a diagram of a non-limiting aspect or embodiment of components of one or more devices and/or one or more systems of FIG. 1;

FIG. 3 is a flowchart of a non-limiting aspect or embodiment of a process for determining a care provider based on a care request;

FIG. 4 is a diagram of a non-limiting aspect or embodiment of a system for content generation for care recommendations;

FIG. 5 is a diagram of a non-limiting aspect or embodiment of a system for training a caregiver matching model of FIG. 4;

FIG. 6 is a flowchart of a non-limiting aspect or embodiment of a process for generating a caregiver matching model; and

FIG. 7 is a flowchart of a non-limiting aspect or embodiment of a process for determining a care provider based on a care request.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. In addition, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if one or more intermediary units (e.g., a third unit located between the first unit and the second unit) processes information received from the first unit and transmits the processed information to the second unit. In some non-limiting embodiments or aspects, a message may refer to a network packet (e.g., a data packet and/or the like) that includes data.

As used herein, the term “server” may refer to one or more devices, such as processors, storage devices, and/or similar components that communicate with client devices and/or other devices over a network, such as the Internet or private networks and, in some examples, provides and facilitates communication among other servers and/or client devices.

As used herein, the term “system” may refer to one or more devices or combinations of devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function. As used herein, the term “real-time” may refer to immediate, near-immediate, or substantially immediate processing and response to input data as it is received, enabling systems or processes to operate with minimal delay, in some examples, the system continuously updates its outputs, predictions, or decisions based on the latest available information, for timely and relevant results.

As used herein, the term “optimize” may refer to the process of improving the efficiency, effectiveness, or performance of caregiving resource allocation and related systems. This involves leveraging machine learning models and real-time feedback to dynamically adjust variables such as caregiver assignments, workload distribution, and content recommendations. The goal is to achieve the best possible outcomes, such as minimizing delays, enhancing caregiver-patient matches, reducing stress, and improving overall system responsiveness, while conserving computational and network resources.

As used herein, the term “supervised learning” may refer to one or more machine learning algorithms that start with known input variables (x₁, x₂, x₃, . . . xₙ), and an output variable (y), and learn the mapping function from the input to the output. The input variables, output variables and the functions are proprietary which have been selected and tuned over time through the combinations of previous data and clinical/care/phycology experts through the Bayesian methodology. The goal of supervised learning is to approximate the mapping function using Constrained Factorial Designs (see reference) and Response Surface Methodology so that predictions may be made about new input variables (x₁, x₂, x₃, . . . xₙ), which may be used to predict output variables (y) for that data. The process of a supervised algorithm learning from the training dataset may be thought of as a teacher supervising the learning process. The correct answers are known by the teacher, the algorithm iteratively makes predictions on the training data and is corrected by the teacher. Learning stops when the algorithm achieves an acceptable level of performance. using ANOVA (Analysis of Variances) for Goodness of Fit. Supervised learning problems may be further grouped into polynomial regression problems and classification problems. For example, a polynomial regression problem involves predicting numerical values such as the reduction in emergency visits. This prediction may rely on historical data like # of comorbidities of the patients, caregiver stress levels, # of ticks in the care check list, medication adherence rate in the past month etc. Response Surface Methodology techniques such as Steepest Ascent Path are used for iterative optimization. Polynomial Regression algorithms like linear and higher order polynomial regression models or decision trees and/or the like, used to forecast such numerical response function. In contrast, classification problems involve assigning input data points to specific categories. An exemplary classification problem may involve sorting care requests into categories such as “high priority,” “standard,” or “low priority.” Classification algorithms like logistic regression, decision trees, or support vector machines may be used, leveraging features such as care request urgency, patient health conditions, and provider specialization to improve the accuracy of care provider assignments. Thereby, learning from historical data and improving care provider matching, resource allocation, and patient experience efficiency.

As used herein, the term “unsupervised learning” may refer to algorithms that work with input variables (x₁, x₂, x₃, . . . xₙ) but no corresponding output variables. The goal of unsupervised learning is to model the underlying structure or distribution in the data, allowing the system to learn more about it. Unlike supervised learning, there are no correct answers or teacher oversight in unsupervised learning. These algorithms are used to discover and present interesting structures or patterns in the data. Unsupervised learning problems may be further grouped into clustering and association rule learning problems. A clustering problem involves identifying inherent groupings in datasets. For example, grouping care providers based on performance metrics like appointment completion rate, patient satisfaction scores, and feedback patterns. An association rule learning problem focuses on uncovering rules that describe relationships in large datasets. For example, rules linking care provider performance to patient outcomes, prioritizing a care provider having high ratings for chronic condition management for a patient having a long-term illness, and prioritizing this provider for matching. Unsupervised learning may identify caregiver stress response triggers. For example, a caregiver feedback indicating stress levels increasing beyond a threshold while the patient has multiple care requests, may prompt stress management suggestions or adaptive workload adjustments.

As used herein, the term “training” refers to the process of analyzing training data to generate a model (e.g., a machine learning algorithm, a prediction model, a classification model, etc.). For example, a training server may use historical non patient identifiable data such as caregiver stress scores, # of medications of the patients, # of comorbidities of the patients, # of times to the bathroom during night hours, caregiver feedback, and real-time feedback from care recipients to train models. These models could be used to predict patient satisfaction, # of hospitalization episodes, enhance caregiver-patient matching, or detect inefficiencies in caregiving workflows. Additionally, such models may help identify caregiver stress levels or predict potential burnout, enabling proactive intervention and improved care outcomes. In some non-limiting embodiments or aspects, a training dataset may be provided from various sources, including caregiver feedback data, patient health assessments, and real-time performance metrics. Deep learning neural network models learn by iteratively mapping inputs to outputs using a training dataset of examples. The training process involves fitting parameters (e.g., weights of connections between neurons in the artificial neural network, etc.) in the neural network until it achieves optimal performance for a specific problem. For example, caregiving session data may be used to generate input vectors that are then used to generate output vectors, including care satisfaction ratings, predicted patient health improvements, and/or the like, which may be scored against expected outcomes. This training process may be repeated numerous times to continually refine and adjust the model's parameters, ensuring it may accurately handle dynamic caregiving scenarios. The training dataset may include historical caregiving interactions from specific regions, collected over predefined periods (e.g., 3 months). Data from sessions flagged as outliers or irrelevant (e.g., incomplete care records, specific conditions outside the system's scope, etc.) may be excluded. In some embodiments, weakly-supervised label generation may be used to categorize caregivers based on keywords or feedback patterns, helping to train models such as caregiver performance or stress prediction models. The labeled dataset may then be split into training (80%) and testing (20%) sets to validate the trained model, ensuring its effectiveness in actual caregiving scenarios.

In some non-limiting embodiments or aspects, to ensure validate causation relationships between input variables and output variables can be derived from the data, Constrained Experimental Design methodology, which was developed and published by Box and Hau (the patient applicant) as a new/original statistical methodology (see reference below), is used to design the experiments to eliminate possible confounding so that causation relationship can be established. This is very different from normal Machine Learning approach, which mainly use observational data that can infer at best correlation but not causation relationships.

Machine learning techniques may include both supervised and unsupervised learning, such as decision trees, logistic regressions, artificial neural networks, Bayesian models, hidden Markov models, and association rule learning. These models may be tailored for specific use cases, such as predicting optimal care provider assignments using decision trees based on real-time caregiver availability. Estimating patient risk levels for delayed care with Bayesian models. Identifying hidden patterns in caregiver feedback data using artificial neural networks.

As used herein, the term “machine learning inference engine” refers to the execution of trained machine learning models to generate an inference output in real-time. An inference engine may leverage various processing units (e.g., CPUs, GPUs, TPUs) to execute prediction models, such as identifying potential gaps in caregiving plans or suggesting optimal adjustments for provider workloads. In some embodiments, the inference engine incorporates feedback from care recipients or caregivers, such as ratings and stress reports, to dynamically refine decision models and improve service delivery. The system includes mechanisms to integrate feedback into its care allocation workflows. For example, after a caregiving session, the system may receive performance ratings from patients or caregivers, including metrics such as care quality, stress levels, and satisfaction. This feedback informs the system's scoring and provider recommendation algorithms, enabling real-time recalibration to ensure optimal care decisions.

As used herein, the term “call-to-action” refers to specific system prompts designed to alert users (e.g., care coordinators, caregivers) to take necessary actions. When a care provider receives a consistently low rating, a notification prompts the care manager to reassess the provider's workload or performance. When caregiver stress exceeds a predefined threshold, the system suggests stress management resources or alternate care strategies.

The term “diagnosis” refers to the capability of analyzing and assessing the operating parameters of the caregiving system. The system dynamically evaluates these parameters, identifying inefficiencies such as delayed responses to care requests, caregiver overload, or mismatched care assignments. It correlates critical caregiving metrics with operational data to flag potential issues proactively. Provided are improved systems, methods, and computer program products for determining a care provider based on a care request. In some non-limiting embodiments or aspects, systems, methods, and computer program products may include receiving data associated with a care request, the data associated with the care request transmitted by a device associated with a care recipient; determining one or more values corresponding to one or more parameters of the care request; determining one or more weighted scores corresponding to one or more care providers based on the one or more values corresponding to the one or more parameters of the care request; and selecting a care provider from among the one or more care providers based on the one or more weighted scores.

By virtue of implementation of the systems, methods, and computer program products described herein, an individual that is able to care for a care recipient based on a request from the care recipient for services may be selected where the individual is able to accommodate the request from the care recipient for services quicker than an individual selected by the care recipient. As a result, the care recipient may not need to wait until the individual caring for the care recipient is available, resulting in a potential reduction in delay in the care recipient receiving services from an individual that is otherwise capable of providing the services requested. Further, systems, methods, and computer program products described herein may result in more accurate selection of individuals able to care for a care recipient than may be selected by the care recipient, leading to a reduction in the consumption of network resources.

In some non-limiting embodiments, computer-implemented methods, systems, and computer program products are disclosed for dynamically optimizing caregiving resource allocation. For example, a method for adaptive care provider assignment may include receiving a plurality of care requests from care recipients, determining a set of care provider attributes including availability, specialization, and current workload, and generating weighted scores for each care provider based on these attributes. The method may further include selecting an optimal care provider for each care request based on the weighted scores, updating the provider's workload upon assignment, and monitoring real-time feedback from both care providers and recipients to recalibrate future assignments dynamically. The system may continuously refine its assignment logic to enhance care efficiency and satisfaction while minimizing delays and resource imbalances.

In this way, non-limiting embodiments of the present disclosure provide an efficient framework for real-time caregiver assignment that reduces the need for manual intervention and improves overall system responsiveness. By leveraging real-time feedback loops, the system can proactively adjust assignments to accommodate shifts in demand or resource availability. Moreover, the system may enhance the accuracy of caregiver matching by incorporating contextual factors such as care urgency, patient history, and caregiver expertise, which may lead to better care outcomes. These improvements may also result in reduced system runtime and network resource consumption by streamlining the decision-making process and minimizing the occurrence of mismatches or reassigned care requests.

Non-limiting embodiments or aspects of the present disclosure are also directed to automated caregiver performance evaluation systems. For example, a method for assessing caregiver performance may include aggregating feedback data from multiple caregiving sessions, analyzing key performance metrics such as task completion rates, patient satisfaction scores, and stress indicators, and generating performance summaries for each caregiver. These summaries may be used to identify top-performing caregivers, highlight areas for improvement, and provide targeted recommendations for stress management or workload adjustments. The system may also use this information to refine caregiver-patient matching algorithms, ensuring that caregivers are assigned to tasks that align with their strengths and areas of expertise.

In some non-limiting embodiments, systems and methods are disclosed for generating and dynamically updating a caregiving taxonomy to provide personalized caregiver content recommendations. For example, a computer-implemented method for caregiver content classification may include receiving caregiver interaction data, determining a plurality of caregiving categories based on predefined caregiving domains, and associating each caregiving category with a set of subcategories tailored to specific tasks or skills. The method may further include identifying a plurality of seed words corresponding to caregiving tasks within the taxonomy and using these seed words to label caregiving content automatically. As caregivers interact with the system and provide feedback, the system refines its taxonomy by introducing new seed words and subcategories aligned with emerging caregiving needs, ensuring content recommendations remain relevant and contextually appropriate.

In this way, by dynamically integrating real-time caregiver data with machine learning models, the disclosed invention leverages a novel approach that improves upon traditional methods by applying advanced machine learning techniques such as artificial neural networks. The systems and methods described herein, identify and remediate inefficiencies in network operations through real-time adjustments. The systems and methods address caregiving processes by automatically detecting and addressing inefficiencies, caregiver stress, or mismatches in care assignments. The technical improvements, including dynamic feedback integration and adaptive taxonomy updates, demonstrate practical applications and transform caregiving resource management systems and methods.

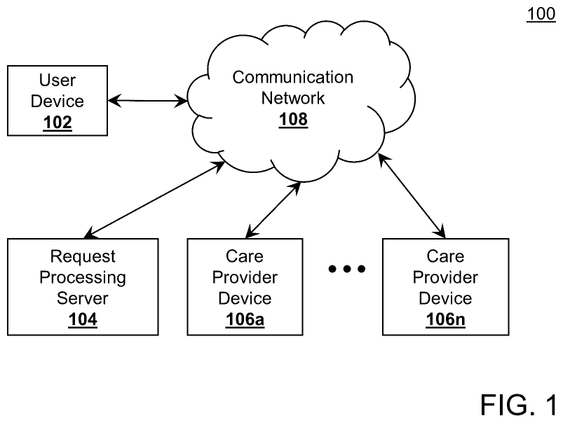

Referring now to FIG. 1, FIG. 1 is a diagram of an example system 100 in which devices, systems, methods, and/or products described herein may be implemented. As shown in FIG. 1, system 100 includes user device 102, request processing server 104, and/or care provider devices 106a-106n (referred to collectively as “care provider devices 106” and individually as “care provider device 106”). User device 102, request processing server 104, and/or care provider devices 106 may interconnect (e.g., establish a connection to communicate, and/or the like) via wired connections, wireless connections, or a combination of wired and wireless connections.

User device 102 may include a device configured to be in communication with request processing server 104 and/or care provider devices 106 via communication network 108. For example, user device 102 may include a mobile device (e.g., a smartphone, a tablet, and/or the like), a laptop computer, a desktop computer, and/or the like. In some non-limiting embodiments or aspects, user device 102 may be associated with a care recipient as described herein.

Request processing server 104 may include one or more devices configured to be in communication with user device 102 and/or care provider devices 106 via communication network 108. For example, request processing server 104 may include a server, a group of servers, and/or the like. In some non-limiting embodiments or aspects, request processing server 104 may be associated with a request processer (e.g., an entity that coordinates the provision of services between care providers and care recipients).

Care provider devices 106 include devices configured to be in communication with user device 102, request processing server 104, and/or other care provider devices 106 via communication network 108. For example, care provider devices 106 may include a mobile device (e.g., a smartphone, a tablet, and/or the like), a laptop computer, a desktop computer, and/or the like. In some non-limiting embodiments or aspects, care provider devices 106 may be associated with a care provider as described herein.

Communication network 108 may include one or more wired and/or wireless networks. For example, communication network 108 may include a cellular network (e.g., a long-term evolution (LTE) network, a third generation (3G) network, a fourth generation (4G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

In some non-limiting embodiments or aspects, the data may include attributes of the care recipient, such as medical conditions, location, preferences, and historical feedback. Request processing server 104 processes this information to extract relevant parameters of the care request, such as urgency, care complexity, and specific patient needs.

In some non-limiting embodiments or aspects, request processing server 104 generates or determines machine learning algorithms to determine weighted scores for potential care providers. These scores are calculated by comparing the care recipient's attributes with care provider attributes, including specialization, location, availability, and historical performance metrics. The request processing server 104 may apply supervised learning techniques to train models using historical care data and real-time feedback, while unsupervised learning methods help uncover patterns in caregiver performance and stress levels.

Once weighted scores are generated, the system automatically selects the most appropriate care provider. This selection ensures an optimized match between the care recipient and the care provider, improving the likelihood of positive outcomes. For example, the system may prioritize care providers who excel in managing chronic conditions or who have received high satisfaction scores for similar care requests. Additionally, the system considers real-time indicators, such as caregiver stress levels, to prevent burnout and ensure sustained quality of care.

Upon selecting a care provider, request processing server 104 generates a care request and transmits it to the selected provider's device (e.g., care provider device 106). The care provider reviews and accepts the request through their device, allowing for seamless coordination and timely care delivery.

The system 100 further includes a feedback loop, collecting data from both care providers and care recipients post-service. This feedback is used to refine the system's machine learning models, dynamically adjusting weighted scores and improving future care provider assignments. For instance, feedback indicating elevated caregiver stress or patient dissatisfaction triggers recalibrations in the provider's weighted score, ensuring the system proactively addresses potential issues.

In some non-limiting embodiments or aspects, system 100 prevents unnecessary hospitalizations by matching patients with caregivers who can address early signs of deteriorating health. The system optimizes care provider selection, reducing patient anxiety and improving satisfaction by ensuring that caregivers are well-suited to meet their specific needs. Additionally, caregiver stress and performance monitoring help maintain high service quality and avoid caregiver burnout, ultimately enhancing the overall efficiency and reliability of caregiving services.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally, or alternatively, a set of systems or a set of devices (e.g., one or more systems, one or more devices) of system 100 may perform one or more functions described as being performed by another set of systems or another set of devices of system 100.

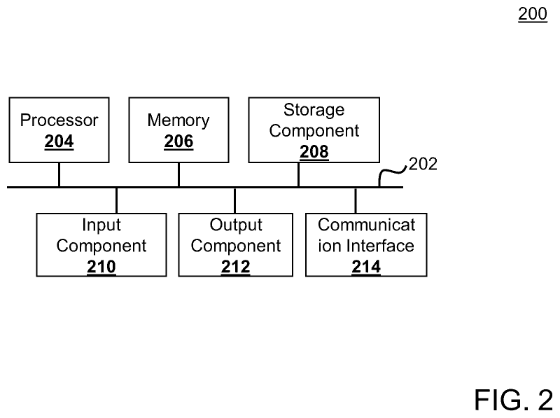

Referring now to FIG. 2, illustrated is a diagram of example components of device 200. As shown in FIG. 2, device 200 may include bus 202, processor 204, memory 206, storage component 208, input component 210, output component 212, and communication interface 214. Device 200 may correspond to one or more devices of user device 102, one or more devices of request processing server 104, one or more devices of care provider devices 106, and/or one or more devices of communication network 108. In some non-limiting embodiments or aspects, one or more devices of user device 102, one or more devices of request processing server 104, one or more devices of care provider devices 106, and/or one or more devices of communication network 108 may include one or more devices 200 and/or one or more components of device 200.

Bus 202 may include a component that permits communication among the components of device 200. In some non-limiting embodiments or aspects, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that may be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, and/or the like). Device 200 may be capable of receiving information from, storing information in, communicating information to, or searching information stored in the data storage or one or more data structures in memory 206 and/or storage component 208. For example, the information may include input data, output data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

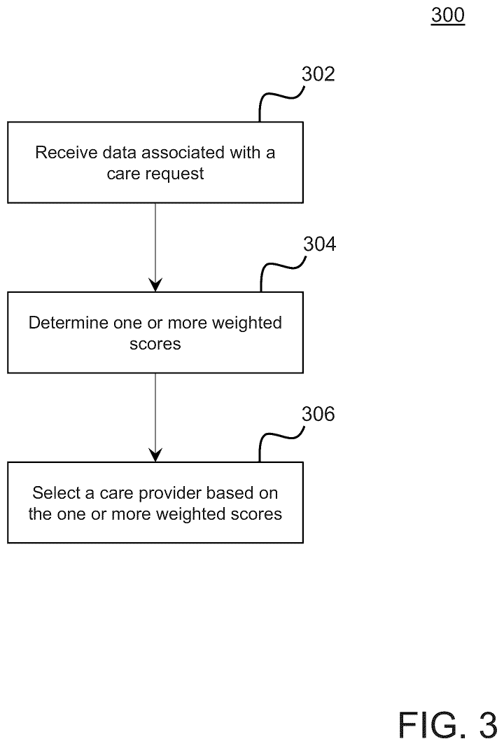

Referring now to FIG. 3, illustrated is a flowchart of a non-limiting aspect or embodiment of a process 300 for determining a care provider based on a care request. In some non-limiting embodiments or aspects, one or more of the functions described with respect to process 300 may be performed (e.g., completely, partially, etc.) by request processing server 104. In some non-limiting embodiments or aspects, one or more of the steps of process 300 may be performed (e.g., completely, partially, and/or the like) by another device or a group of devices separate from and/or including request processing server 104, such as user device 102 and/or one or more care provider devices 106.

As shown in FIG. 3, at step 302, process 300 may include receiving data associated with a care request. For example, request processing server 104 may receive data associated with a care request. In such an example, the care request may represent a request by a care recipient to schedule one or more services with one or more care providers. In some non-limiting embodiments or aspects, request processing server 104 may receive data associated with the care request from user device 102. For example, request processing server 104 may receive data associated with the care request from user device 102 based on user device 102 receiving input. In such an example, user device 102 may receive input from a care recipient, and user device 102 may generate and transmit the data associated with the care request to request processing server 104 based on the input received from the care recipient.

In some non-limiting embodiments or aspects, request processing server 104 may receive data associated with one or more attributes of a care recipient. For example, request processing server 104 receives care request data associated with a plurality of care requests involving a plurality of care recipients and a plurality of care providers. Care request data may include identification of care recipients, care provider information (e.g., care provider ID or token), location, required time intervals, and specific care request attributes. For example, request processing server 104 may receive data associated with one or more attributes of a care recipient from user device 102. In such an example, request processing server 104 may store the data associated with the one or more attributes of the care recipient in a data structure (e.g., a database) and request processing server 104 may retrieve the data associated with the one or more attributes of the care recipient from the data structure based on (e.g., in response to) receiving data associated with a care request, as described above. Additionally, or alternatively, request processing server 104 may receive the data associated with the one or more attributes of a care recipient from care provider devices 106. For example, request processing server 104 may receive the data associated with the one or more attributes of the care recipient from one or more care provider devices 106 and request processing server 104 may store the data associated with the one or more attributes of the care recipient in a data structure. In such an example, request processing server 104 may retrieve the data associated with the one or more attributes of the care recipient from the data structure based on (e.g., in response to) receiving data associated with a care request, as described above. In some non-limiting embodiments or aspects, the data associated with the one or more attributes of the care recipient may include one or more of data associated with an identifier of the care recipient (e.g., a unique identifier corresponding to the care recipient, a driver's license number corresponding to the care recipient, and/or the like), data associated with a location that is associated with the care recipient (e.g., a home address of the care recipient, a work address of the care recipient, and/or the like), data associated with a date and/or frequency for a care request (e.g., a day, a week, a month, and/or the like), data associated with a requested start time for a care request (e.g., a request to start treatment in the morning or afternoon, a request to start treatment between one or more hours of the day, and/or the like), data associated with a requested end time for a care request (e.g., a request to end treatment by the morning or afternoon, a request to end treatment by one or more hours of the day, and/or the like), an indication of whether the care request is a one-time care request or a repeating care request, data associated with one or more services involved in a care request (e.g., a specification of one or more services to be performed), and data associated with one or more demographic requests that are associated with the care request (e.g., a request that the gender of the care provider match the gender of the care recipient, a request that the age of the care provider be within a certain range of ages, and/or the like). In some non-limiting embodiments or aspects, request processing server 104 may determine one or more values corresponding to the one or more parameters of the care request. For example, request processing server 104 may determine one or more values corresponding to the one or more parameters of the care request based on the data associated with the care request.

In some non-limiting embodiments or aspects, request processing server 104 may receive data associated with one or more rules associated with one or more preferences of a care recipient. For example, request processing server 104 may receive data associated with one or more rules associated with one or more preferences of a care recipient involved in a care request as described herein. In some non-limiting embodiments or aspects, request processing server 104 may receive the data associated with the one or more rules associated with one or more preferences of the care recipient based on request processing server 104 transmitting a request to user device 102. For example, request processing server 104 may receive the data associated with the one or more rules associated with one or more preferences of the care recipient based on request processing server 104 transmitting a request to user device 102, where the request includes a request for input from the care recipient specifying the one or more rules associated with the one or more preferences of the care recipient. In some non-limiting embodiments or aspects, the one or more rules may include, for example, a rule that the gender of a care provider selected matches the gender of the care recipient, a rule that the care provider selected be associated with an address within a predetermined distance of the address associated with the care recipient, and/or the like.

As shown in FIG. 3, at step 304, process 300 may include determining one or more weighted scores. For example, request processing server 104 may determine one or more weighted scores. In some non-limiting embodiments or aspects, request processing server 104 may determine the one or more weighted scores for one or more care providers based on the one or more values corresponding to the one or more parameters of the care request. For example, request processing server 104 may determine the one or more weighted scores for the one or more care providers based on the one or more values corresponding to the one or more parameters of the care request.

In some non-limiting embodiments or aspects, request processing server 104 may compare one or more weighted scores to one or more attributes of one or more care providers. For example, request processing server 104 may compare values of the one or more weighted scores to values of the one or more attributes of the one or more care providers. In such an example, request processing server 104 may determine the one or more weighted scores based on request processing server 104 comparing the values of the one or more weighted scores to the values of the one or more attributes of the one or more care providers. In such an example, the weighted scores may reflect dynamically updated correlations between care recipient needs and care provider characteristics, including care provider operational features, care recipient embedded features, and care provider embedded features. For example, care recipient embedded features may include correlations between care recipient preferences (e.g., preferred care style, cost preferences) and care provider attributes, while care provider embedded features may link provider-specific qualities to historical care interactions.

In some non-limiting embodiments or aspects, request processing server 104 may transmit data associated with the one or more weighted scores corresponding to the one or more care providers to user device 102. For example, request processing server 104 may transmit data associated with the one or more weighted scores corresponding to the one or more care providers to user device 102 to enable user device 102 to generate a display including the one or more scores of the one or more care providers. In some non-limiting embodiments or aspects, user device 102 may receive input indicating a selection of a care provider. For example, user device 102 may receive input indicating a selection of a care provider based on user device 102 generating a display including the one or more scores of the one or more care providers. In such an example, user device 102 may transmit data associated with the input to request processing server 104.

In some non-limiting embodiments or aspects, request processing server 104 may select a care provider from among one or more care providers based on one or more rules of a care recipient. For example, request processing server 104 may select a care provider from among one or more care providers based on one or more rules of a care recipient involved in a care request. In such an example, request processing server 104 may select the care provider from among the one or more care providers based on request processing server 104 comparing the one or more rules of the care recipient involved in the care request to the one or more attributes of the care provider in addition to, and/or in the alternative to, the one or more weighted scores of the one or more care providers. In some non-limiting embodiments or aspects, request processing server 104 may select a care provider based on the input received at user device 102. For example, request processing server 104 may select a care provider based on the input received at user device 102 indicating the selection of the care provider. In such an example, a care provider matching model may predict care provider recommendations by determining a combination of care provider operational features (e.g., availability, specialization), care recipient embedded features, and care provider embedded features using a deep learning neural network.

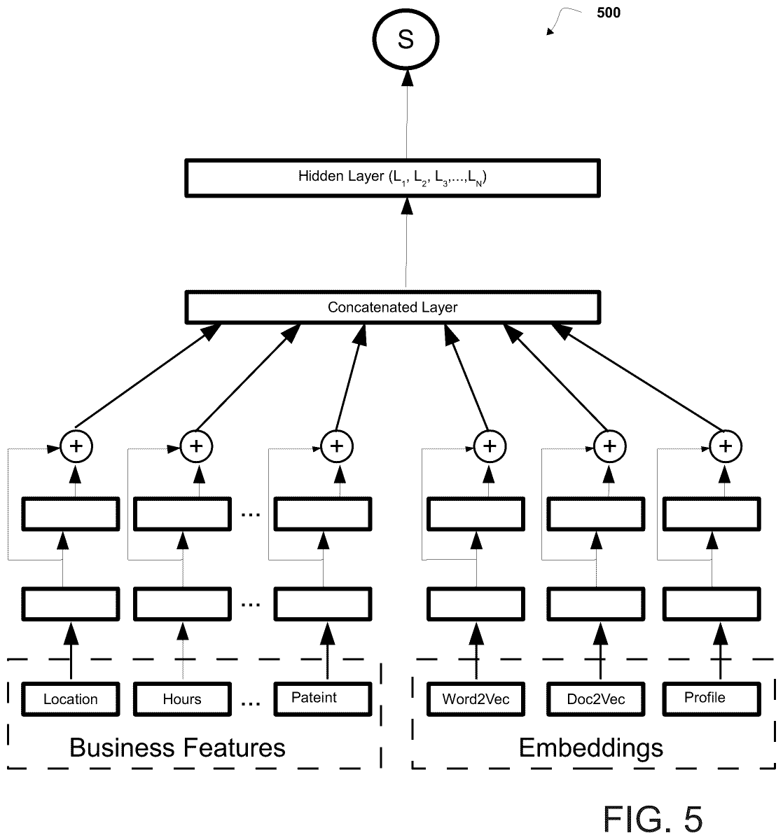

In some non-limiting embodiments or aspects, request processing server 104 may determine one or more weighted scores based on data associated with a rating of one or more care providers. For example, request processing server 104 may determine one or more weighted scores based on data associated with a rating of one or more care providers that may be involved in a care request. In some non-limiting embodiments or aspects, request processing server 104 may receive the data associated with a rating of one or more care providers from user device 102. For example, user device 102 may receive input from a care recipient associated with user device 102, the input specifying the rating of the one or more care providers. In such an example, user device 102 may transmit data associated with the rating of the one or more care providers to request processing server 104. In some non-limiting embodiments or aspects, determining one or more weighted scores includes determining a plurality of care provider classifications. For example, care provider matching system 102 determines a plurality of care provider classifications based on care provider attributes associated with the plurality of care requests as shown below with reference to FIG. 5.

In some non-limiting embodiments or aspects, a caregiving taxonomy for content classification includes categories designed to comprehensively address the full spectrum of caregiving responsibilities. These categories may encompass all caregiving stages, from initial training and preparation to advanced caregiving techniques for specific medical conditions. In some embodiments, the caregiving taxonomy is structured to prioritize categories based on the caregiver's immediate needs and preferences, which may be dynamically adjusted in response to new interaction data.

For example, the taxonomy may include broad caregiving categories such as stress management and patient care training, with each category further divided into subcategories that target specific tasks or skills. Stress management might include guided meditation and breathing exercises, while patient care training could involve instructional content on feeding, toileting, and mobility assistance.

In other embodiments, the taxonomy may be selectively reduced to focus only on a subset of caregiving tasks relevant to the caregiver's current caregiving scenario. For example, for a caregiver looking after a patient recovering from a stroke, the system may prioritize content subcategories such as mobility assistance, fall prevention, and communication techniques tailored to post-stroke care.

This taxonomy ensures that caregivers receive content aligned with their immediate context, helping them navigate complex caregiving scenarios more effectively. Moreover, the taxonomy may be further fine-tuned to cover specific caregiving scenarios, such as Alzheimer's care, post-surgical recovery, or pediatric care for chronic illnesses, providing highly targeted and relevant content.

TABLE 1

| Caregiving Category | Related Subcategories |
|---|---|
| Stress Management | Guided Meditation, Breathing Exercises |
| Patient Care Training | Feeding, Toileting, Mobility Assistance |
| Self-Care Techniques | Exercise, Nutrition, Relaxation |
| Communication Skills | Patient Interaction, Family Counseling |
| Emergency Preparedness | CPR, Fall Response, Medical Alerts |

The caregiving taxonomy also provides personalized recommendations, as caregivers may explore resources within categories and subcategories that resonate most with their caregiving journey. This modular structure ensures flexibility, adaptability, and relevance.

In some non-limiting embodiments or aspects, caregiver label generator 104 is configured to determine a plurality of seed words that serve as foundational elements for labeling caregiving content. These seed words are derived from established caregiving domains and represent core tasks, skills, or needs within the caregiving taxonomy. For example, seed words associated with stress management might include “meditation,” “breathing”, “relaxation,” and/or the like, while seed words for patient care training might encompass “feeding,” “mobility,” and “hygiene.”

The system leverages these seed words to categorize content automatically and consistently. By using machine learning techniques, caregiver label generator 104 identifies patterns in caregiver interaction data, associating specific keywords with corresponding caregiving tasks or content types.

In some embodiments, the seed words are not static but evolve based on continuous feedback from caregivers. As caregivers interact with the system and provide input on the relevance or effectiveness of content, new keywords may be introduced to reflect emerging caregiving needs or preferences. For example, if multiple caregivers provide feedback highlighting the importance of “digital communication tools” for remote caregiving, these terms may be incorporated as seed words in the communication skills category. For example, if multiple caregivers provide feedback emphasizing the value of “progressive muscle relaxation” as a stress-relief technique, this phrase may be incorporated as a seed word within the Stress Management category, ensuring that related content such as guided exercises or instructional videos becomes more prominently recommended. For example, if caregivers frequently highlight the need for “fire safety protocols” in their feedback, particularly for homes with mobility-impaired patients, these terms could be added as seed words under the Emergency Preparedness category, prompting the system to prioritize content such as evacuation plans and fire drill guides. For example, if feedback reveals a growing interest in “mindful eating practices” as a component of caregiver self-care, the system could add this phrase as a seed word under the Self-Care Techniques category, expanding content suggestions to include dietary mindfulness strategies and healthy meal planning. For example, if caregivers indicate the importance of “wound care management” for patients recovering from surgery, these terms could be introduced as seed words under the Patient Care Training category, ensuring resources like dressing change tutorials and infection prevention tips are easily accessible. For example, if feedback suggests that “non-verbal communication” is a critical skill for caregivers of non-verbal or cognitively impaired patients, this term could be integrated as a seed word under the Communication Skills category. This would help prioritize content that teaches caregivers how to interpret body language, facial expressions, and other non-verbal cues.

In some non-limiting embodiments or aspects, the system employs natural language processing (NLP) techniques to enhance the accuracy and relevance of seed word selection. NLP algorithms analyze caregiver feedback, comments, and engagement patterns to identify potential new seed words. This approach ensures that the caregiving taxonomy remains current and responsive to the evolving landscape of caregiving practices.

In some non-limiting embodiments or aspects, seed words also play a critical role in enabling multilingual support and localization. For example, seed words may be translated or culturally adapted to align with regional caregiving norms and language preferences, ensuring the system remains accessible and effective for caregivers in diverse settings. Seed words may be translated or culturally adapted to align with regional caregiving norms and language preferences, ensuring the system remains accessible and effective for caregivers in diverse settings. This means that key terms or phrases used to categorize caregiving content may be adjusted to reflect the linguistic and cultural nuances of different regions or communities, maintaining the system's relevance and usability.

For example, in some non-limiting embodiments, seed words may be translated to accommodate caregivers who speak different languages. Translation for Language Preferences could involve converting terms like “stress management” and “breathing exercises” into “manejo del estrés” and “ejercicios de respiración” for Spanish-speaking users. This ensures that the caregiving content remains linguistically understandable while retaining its intended meaning and application.

In other embodiments or aspects, seed words may undergo cultural adaptation for norms and practices to better reflect regional caregiving customs. For example, in cultures where caregiving is a collective responsibility involving extended family, seed words like “family counseling” might be adapted to emphasize “multigenerational support.” Similarly, stress management content might incorporate region-specific techniques, such as yoga in South Asia or tai chi in East Asia, aligning with local practices and expectations.

Additionally, seed words may be aligned with Regional Caregiving Norms to address specific caregiving needs prevalent in different areas. For example, in countries where caregiving typically occurs at home, seed words like “fall prevention” might include resources on home safety modifications, such as installing handrails or using non-slip mats. In regions with limited access to healthcare facilities, content might prioritize terms like “basic first aid” or “telemedicine tools,” ensuring caregivers have the resources they need to manage care effectively.

In some embodiments, Localizing Emergency Preparedness involves tailoring seed words to reflect region-specific disaster risks. For example, in earthquake-prone areas, seed words like “disaster response” might emphasize “earthquake preparedness” (e.g., “drop, cover, and hold”), while in hurricane-prone areas, content might highlight “hurricane evacuation plans” and emergency supplies.

In some non-limiting embodiments or aspects, these adaptations preserve caregiver accessibility across diverse contexts. By translating and culturally adapting seed words, the system ensures that caregiving content is not only linguistically accessible but also culturally relevant. This enhances caregiver engagement, builds trust, and ensures the platform delivers effective, contextually appropriate support to users, regardless of their geographical or cultural background.

In some non-limiting embodiments or aspects, by integrating seed words with dynamic classification models, caregiver label generator 104 provides a robust framework for content categorization, helping caregivers quickly find the resources most relevant to their unique caregiving challenges.

In some non-limiting embodiments or aspects, request processing server 104 may receive data associated with the one or more attributes of the one or more care providers. For example, request processing server 104 may receive data associated with the one or more attributes of the one or more care providers based on request processing server 104 determining the one or more weighted scores. In some non-limiting embodiments or aspects, request processing server 104 may receive data associated with the one or more attributes of the one or more care providers, the data associated with the one or more attributes of the one or more care providers including data associated with an identifier of a care provider (e.g., a unique identifier for each care provider), data associated with a location of a care provider (e.g., data associated with a commercial address of a care provider, data associated with a residential address of a care provider, and/or the like), data associated with an availability of a care provider (e.g., one or more days and/or times of day that a care provider is available to provide services), data associated with one or more demographics of a care provider (e.g., an age range of the care provider, a gender of the care provider, and/or the like), data associated with one or more qualifications of a care provider, and/or the like. In some non-limiting embodiments or aspects, the one or more qualifications of the care provider may include a level of experience associated with one or more corresponding care providers, one or more fields (e.g., nursing, primary care physician, cardiologist physician, and/or the like) that one or more care providers have experience in, one or more times (e.g., one or more periods of time such as a month, a year, a plurality of years, and/or the like) associated with an amount of time the one or more care providers have had experience with the one or more fields, one or more certifications attained by a care provider, one or more licenses attained by a care provider, one or more ratings of a care provider, and/or the like. In some non-limiting embodiments or aspects, the data associated with the one or more attributes of the one or more care providers may include data associated with a care provider wellness score representing the mental health and/or wellbeing of one or more care providers.

In some non-limiting embodiments or aspects, request processing server 104 may determine the one or more attributes of the one or more care providers. For example, request processing server 104 may determine the one or more attributes of the one or more care providers based on request processing server 104 transmitting data associated with one or more qualifications to care provider devices 106. In such an example, the data associated with one or more qualifications to care provider devices 106 may include one or more training videos, one or more invitations to communicate with a different care provider (e.g., a coach that assists care providers who are training to provide one or more services) associated with care provider device 106 that is associated with a different care provider, and/or the like. In some non-limiting embodiments or aspects, care provider devices 106 that receive the data associated with one or more qualifications may prompt the care provider operating care provider devices 106 to provide input (e.g., responses to questions, and/or the like) indicating that the care providers are capable of providing one or more services. For example, care provider devices 106 that receive the data associated with one or more qualifications to care provider devices 106 may prompt the care provider operating care provider devices 106 to provide input (e.g., responses to questions associated with the qualifications of the one or more care providers, responses to questions about the mental health and/or wellbeing of one or more care providers, and/or the like) indicating that the care providers are capable of providing one or more services. In such an example, care provider devices 106 that receive the input may transmit data associated with the input provided by the care providers to request processing server 104. In some non-limiting embodiments or aspects, request processing server 104 may determine the one or more attributes of the one or more care providers based on the input provided by the care providers, described above. For example, request processing server 104 may determine the one or more attributes of the one or more care providers based on the input provided by the care providers by request processing server 104 comparing the input provided by the care providers to predetermined inputs associated with correct inputs indicating competency to provide the one or more services.

As shown in FIG. 3, at step 306, process 300 may include selecting a care provider based on the one or more weighted scores. For example, request processing server 104 may select a care provider based on the one or more weighted scores. In some non-limiting embodiments or aspects, request processing server 104 may select the care provider based on the one or more weighted scores, where the selected care provider is associated with a weighted score that is higher than one or more other weighted scores of one or more other care providers. Additionally, or alternatively, request processing server 104 may select the care provider based on the one or more weighted scores, where the selected care provider is associated with a weighted score that is lower than one or more other weighted scores of one or more other care providers.

In some non-limiting embodiments or aspects, request processing server 104 may select a care provider based on an amount of credits (e.g., a type of currency such as funds, credits purchased with funds in advance of the transmission of the data associated with the care request from user device 102, and/or the like). For example, request processing server 104 may select a care provider based on an amount of credits purchased by the care recipient. Additionally, or alternatively, request processing server 104 may select a care provider based on the amount of credits attained by the care recipient where the care recipient is a care provider. For example, request processing server 104 may select a care provider based on the amount of credits attained by the care recipient where the care recipient is a care provider where the amount of credits attained by the care recipient is sufficient to satisfy the amount of credits needed to obtain the services included in the care request from the care provider that was selected. In some non-limiting embodiments or aspects, request processing server 104 may maintain one or more accounts for one or more care recipients and/or one or more care providers. For example, request processing server 104 may maintain one or more accounts for one or more care recipients and/or one or more care providers based on one or more care requests. In such an example, request processing server 104 may maintain one or more accounts for one or more care recipients and/or one or more care providers based on one or more care requests by adding and/or subtracting credits from the one or more accounts for one or more care recipients and/or one or more care providers based on the acceptance and/or completion of services associated with the one or more care requests. In some non-limiting embodiments or aspects, the one or more accounts of the one or more care recipients may be associated with one or more individuals associated with the one or more care recipients (e.g., a parent, a spouse, a child, a guardian, a governmental agency maintaining and/or providing benefits on behalf of the care recipient, a non-profit organization providing benefits on behalf of the care recipient, and/or the like). In some non-limiting embodiments or aspects, an account associated with a care provider may also be associated with a care recipient where the care provider also is involved in one or more care requests as a care recipient.

In some non-limiting embodiments or aspects, request processing server 104 may generate a care request for a care provider. For example, request processing server 104 may generate a care request for a care provider based on request processing server 104 selecting a care provider. In some non-limiting embodiments or aspects, request processing server 104 may generate the care request for the care provider, where the care request includes data associated with the care request received from user device 102. Additionally, or alternatively, request processing server 104 may generate the care request for the care provider, where the care request includes data associated with the care request that is configured to cause a device to display an image representing the care request. For example, request processing server 104 may generate the care request for the care provider, where the care request includes data associated with the care request that is configured to cause care provider device 106 to display an image representing the care request based on (e.g., in response to) care provider device 106 receiving the care request.

In some non-limiting embodiments or aspects, request processing server 104 may transmit data associated with the care request. For example, request processing server 104 may transmit data associated with the care request to care provider device 106. In some non-limiting embodiments or aspects, request processing server 104 may transmit the data associated with the care request to care provider device 106, where care provider device 106 is associated with (e.g., operated by) the care provider that was selected. In some non-limiting embodiments or aspects, care provider device 106 may display an image based on care provider device 106 receiving the data associated with the care request. For example, care provider device 106 may display an image based on care provider device 106 receiving the data associated with the care request, where the image represents the care request.

In some non-limiting embodiments or aspects, the data associated with the care request may include data associated with a personalized message. For example, data associated with the care request may include data associated with a personalized message intended for a care provider that may provide one or more services, where the personalized message specifies one or more of the profile of the care recipient (e.g., a profile indicating whether a care recipient is associated with a dementia diagnosis, a profile indicating whether a care recipient is associated with a post stroke diagnosis, a profile indicating the experience and/or stage in caregiving of the care recipient, a profile including a profile associated with the care provider, a profile indicating a time of day that one or more services are to be provided to the care recipient, a profile indicating one or more days of the week that a service is to be provided, a profile indicating one or more stress areas associated with the care recipient, and/or the like). In some non-limiting embodiments or aspects, request processing server 104 may be configured to perform a process for adapting text based gold standard training associated with one entity (e.g., a government, a country, a state, and/or the like) into another specific international entity associated with environments such as, in a non-limiting example, another country adjusted for cultural, regulatory, and economic differences in a multimedia story based delivery of data associated with a care request. In some non-limiting embodiments or aspects, request processing server 104 may be configured to implement one or more business models for delivering training and/or educational content, subscriptions (e.g., paid-for subscriptions), referrals for care providers, reimbursing family caregivers, and/or the like.

In some non-limiting embodiments or aspects, request processing server 104 may store data associated with a library of media to be presented to one or more care providers. For example, request processing server 104 may store data associated with a library of media to be presented to one or more care providers where the library includes, guides (e.g., guides for providing services for a patient and/or to oneself), tips (e.g., tips for improving physical and/or mental health for a patient and/or oneself), educational material, and/or the like. In some non-limiting embodiments or aspects, request processing server 104 may select one or more examples of media from the library of media. For example, request processing server 104 may select one or more examples of media from the library of media to be provided to a care provider. In such an example, request processing server 104 may select one or more examples of media from the library of media. For example, request processing server 104 may select one or more examples of media from the library of media to be provided to a care provider based on request processing server 104 determining that one or more examples of media are more appropriate than one or more other examples of media (e.g., that the one or more examples of media apply to one or more services involving the care provider, that the one or more examples of media may be useful in improving the physical and/or mental health of the care provider, that the one or more examples of media may be useful in reducing the stress of the care provider, and/or the like). In some non-limiting embodiments or aspects, request processing server 104 may transmit data associated with the one or more examples of media from the library of media to be provided to a care provider. For example, processing server 104 may transmit data associated with the one or more examples of media from the library of media to be provided to a care provider to one or more care provider devices 106.

In some non-limiting embodiments, request processing server 104 may determine an amount of credits associated with a care request. For example, request processing server 104 may determine an amount of credits associated with a care request based on the care request. In some non-limiting embodiments, request processing server 104 may determine an amount of credits associated with a care request based on the care request and a predetermined amount of credits associated with the care request. For example, request processing server 104 may determine an amount of credits associated with a care request based on the care request and a predetermined amount of credits associated with the care request, where the predetermined amount of credits associated with the care request corresponds to a predetermined amount of credits for the one or more services specified by the care request. Additionally, or alternatively, request processing server 104 may determine an amount of credits associated with the care request based on an amount of time spent by a care provider providing services specified by the care request. For example, request processing server 104 may determine an amount of credits associated with the care request based on an amount of time spent by a care provider providing services specified by the care request based on request processing server 104 multiplying a credit rate associated with a care provider and an amount of time spent by the care provider providing the services specified by the care request. In such an example, care provider device 106 associated with the care provider providing the services specified in the care request may transmit data associated with the services provided by the care provider specifying the amount of time spent by the care provider providing the services to request processing server 104. Request processing server 104 may then determine the amount of credits associated with the care request. In some non-limiting embodiments, request processing server 104 may transfer the credits associated with a care request from an account associated with the care recipient involved in the care request to an account associated with the care provider. In some non-limiting embodiments or aspects, request processing server 104 may determine the credit rate associated with a care provider based on the one or more attributes of the one or more care providers, a non-limiting example of which is described above. In some non-limiting embodiments or aspects, credits may be withdrawn from one or more accounts and transferred to one or more personal and/or business accounts.

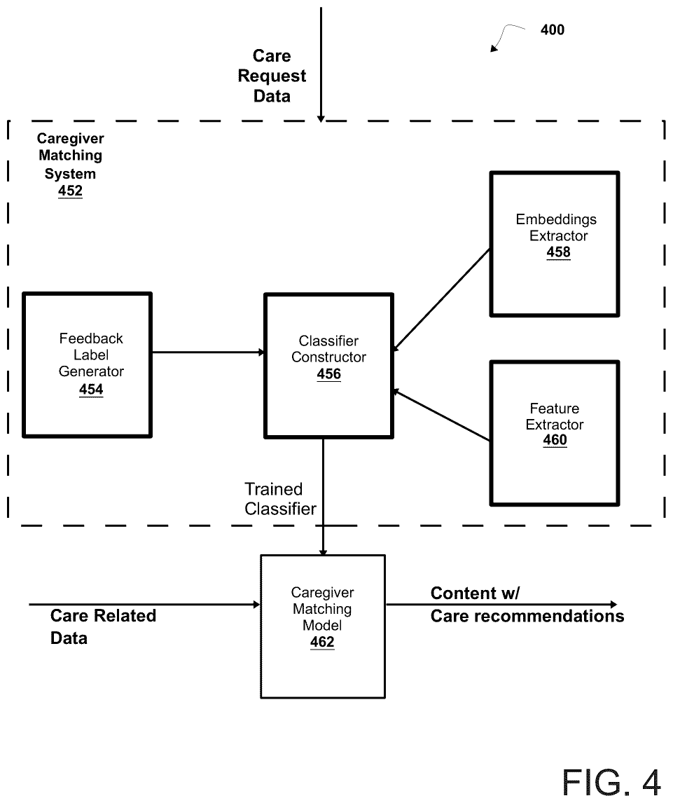

FIG. 4 is a diagram of an example caregiver matching system (CMS) 452 in which the devices, systems, methods, and/or products described herein may be implemented. As shown in FIG. 4, caregiver matching system 452 includes feedback label generator 454, classifier constructor 456, embeddings extractor 458, feature extractor 460, and caregiver matching model 462. These components collectively process caregiver and patient data to provide optimal caregiver-patient pairings. The system provides machine learning to generate real-time, dynamic predictions and recommendations.

In some non-limiting embodiments or aspects, caregiver matching system 452 receives care request data as input, which may include patient-specific information (e.g., care needs, preferences) and real-time caregiver performance data. This data is processed through various system components to generate optimized care recommendations.

In some non-limiting embodiments or aspects, feedback label generator 454 generates feedback labels based on historical and real-time caregiver-patient interaction data. Feedback may include patient satisfaction ratings, caregiver stress levels, and session performance metrics. These labels serve as essential training data for machine learning models.

In some non-limiting embodiments or aspects, classifier constructor 456 obtains and extracts training data from the feedback label generator 454 and integrates additional features extracted from caregiver and patient interactions. For example, classifier constructor 456 builds (e.g., generates, provides, transmits, etc.) predictive models designed to enhance caregiver matching and detect potential inefficiencies, such as caregiver stress or mismatched care assignments.

In some non-limiting embodiments or aspects, embeddings extractor 458 converts high-dimensional data (e.g., caregiver attributes, patient preferences) into numerical vector representations. For example, embeddings identify complex relationships between caregivers and care recipients, improving the accuracy of the matching process.

In some non-limiting embodiments or aspects, feature extractor 460 obtains and processes raw interaction data from caregiving sessions to derive structured features. These features may include metrics like session duration, real-time patient satisfaction scores, and caregiver stress indicators. The structured data serves as input for predictive modeling.

The caregiver matching model 462 leverages trained classifiers to dynamically evaluate and recommend optimal caregiver-patient pairings. The model uses weighted scoring mechanisms based on real-time feedback and other extracted features to predict the likelihood of positive outcomes (e.g., patient satisfaction, caregiver retention). The system can also suggest adjustments to care strategies, ensuring alignment with patient needs and caregiver capabilities.

In some non-limiting embodiments or aspects, data flow and system operation includes input data, embeddings, and a trained classifier. For example, care request data, patient preferences, caregiver availability, and feedback, flow into the system. Data moves through the embeddings extractor 458 and feature extractor 460 to generate meaningful input for model training and prediction. The trained classifier within the caregiver matching model 462 generates real-time caregiver recommendations and care strategies.

The number and arrangement of systems and/or devices shown in FIG. 4 are provided as examples. Additional components, fewer components, or different configurations may be used, depending on specific implementation requirements. For instance, some systems may incorporate additional layers of feedback analysis or include specialized predictive algorithms for unique caregiving scenarios.

Referring now to FIG. 4, illustrated is the caregiver matching system (CMS) 452 in which the devices, systems, methods, and/or products described herein may be implemented. Caregiver Matching System 452 includes several integrated components designed to optimize the caregiver selection process using machine learning and real-time data analysis. As shown in FIG. 4, the system comprises the following elements:

In some non-limiting embodiments or aspects, feedback label generator 454 determines real-time and historical caregiver feedback. This may involve generating labeled data sets based on patient reviews, caregiver performance metrics, and stress indicators. These labeled data sets serve as the foundation for supervised learning in the system. For example, classifier Constructor 456 receives or obtains the labeled data from the feedback label generator 454 to train machine learning models. These models classify caregivers based on their attributes, such as specialization, performance ratings, and patient satisfaction scores. The classifier constructor 456 improves the model's ability to predict optimal caregiver-patient matches.

In some non-limiting embodiments or aspects, embeddings extractor 458 extracts multidimensional embeddings from various data sources, such as caregiver profiles and patient interaction logs. These embeddings, representing caregiver competencies and patient needs, are fed into subsequent layers for deeper analysis.

In some non-limiting embodiments or aspects, feature extractor 460 analyzes caregiver and patient features, including demographic information, care requirements, and stress levels. This component ensures that both quantitative and qualitative data points are included in the caregiver matching process.

In some non-limiting embodiments or aspects, caregiver matching model 462 integrates, adds, removes, or otherwise merges inputs from the other components to predict the best caregiver match. The caregiver matching model 462 may include supervised and unsupervised learning techniques to generate weighted scores, reflecting the likelihood of successful caregiver-patient interactions.

The caregiver matching system 452, as illustrated in FIG. 4, works in conjunction with user device 102, request processing server 104, and care provider devices 106a-106n, as shown in FIG. 1. The system enables real-time caregiver selection by dynamically updating the caregiver matching model 462 based on incoming feedback from care sessions. The process is enhanced by the feedback loop, where post-care feedback continuously refines the model's predictive accuracy.

The CMS 452 enhances care delivery by reducing mismatches, improving patient satisfaction, and preventing caregiver burnout. For example, by leveraging feedback label generator 454 and classifier constructor 456, the system identifies caregivers prone to stress and adapts assignments accordingly, thereby reducing unnecessary hospitalizations and improving overall care efficiency.

With reference to FIG. 5, in some non-limiting embodiments or aspects, a deep learning neural network is used to classify caregivers based on various caregiving-related attributes. As shown in FIG. 5, the neural network includes several parallel input paths, each corresponding to a different feature, such as caregiver-specific features (e.g., caregiver performance metrics, patient feedback, caregiving styles, etc.) and embeddings (e.g., caregiver embeddings, patient embeddings, feedback embeddings, etc.). First, the caregiver classification model receives and passes each of the different features (e.g., in the form of a set of vectors of real numbers) through two hidden layers, where the two layers have residual connections. Next, the model concatenates the resultant layers to create a single concatenated layer. Lastly, the model passes the concatenated layer through two hidden layers and applies a SoftMax activation to generate the output layer.

In some non-limiting embodiments or aspects, a training dataset may be provided from a set of historical caregiving interactions and feedback. Deep learning neural network models learn by iteratively mapping inputs to outputs given a training dataset of examples. The training process involves fitting parameters (e.g., weights of connections between neurons in the artificial neural network, etc.) in the network until it proves effective at solving the specific problem. For example, a classifier constructor receives a training dataset and fits the parameters of the model as shown in FIG. 5, by inputting a first caregiving session as an input vector to produce an output vector (e.g., caregiver performance rating, patient satisfaction level, etc.) that may be compared or scored against a target (e.g., expected satisfaction rating). This process may be repeated numerous times to adjust the parameters of the model continually.

The training dataset may include caregiving sessions across a predefined time period (e.g., 3 months), including various caregiving scenarios and patient conditions (e.g., dementia care, post-stroke recovery, etc.). Caregiving sessions flagged as incomplete or irrelevant may be excluded. In some non-limiting embodiments, weakly-supervised label generation could be used to classify caregivers based on feedback patterns or predefined rules, improving model training accuracy. The labeled dataset may then be split into training (80%) and testing (20%) sets to validate the trained model's accuracy.

In some non-limiting embodiments, a user interface displays (or reports) the declined accuracy scores when removing specific features (e.g., one at a time). For example, caregiver performance metrics and feedback embeddings may offer the most useful features for accurate classification. Among the embeddings, patient feedback and historical caregiving session data provide significant indicators of caregiving outcomes. Additionally, patient profiles, such as medical history and caregiving preferences, play a vital role in improving the model's accuracy. This may improve feature importance and the changes in accuracy.

In some non-limiting embodiments or aspects, the system includes a real-time caregiver matching module that leverages machine learning models to predict the most suitable caregiver for a patient. The model may analyze patient-specific data, such as medical conditions (e.g., dementia, post-stroke recovery, chronic illnesses), caregiving preferences (e.g., preferred communication style, cultural background), and past performance metrics of caregivers (e.g., patient satisfaction scores, on-time service rates, adherence to care protocols). The matching process integrates real-time feedback and dynamically updates recommendations to ensure the best possible caregiver-patient pairing. By prioritizing attributes like patient needs and caregiver strengths, the system enhances care quality and patient outcomes.

In some non-limiting embodiments or aspects, the system may utilize historical feedback data to predict the likelihood of patient satisfaction in specific caregiver-patient pairings. The model analyzes caregiver performance metrics (e.g., care quality scores, responsiveness) alongside patient feedback (e.g., ratings, comments) to generate satisfaction scores for potential pairings. This prediction model may be used proactively to inform caregiver selection, ensuring higher levels of satisfaction. For example, caregivers who have previously performed well in specific care scenarios or with patients with similar profiles may be prioritized for new care requests. This approach fosters better patient experiences and long-term engagement.

In some non-limiting embodiments or aspects, the system includes a caregiver stress and burnout detection system and method. This system uses real-time feedback and session data to classify caregivers based on their likelihood of experiencing stress or burnout. Features (e.g., workload intensity, patient interaction complexity, care session duration, etc.) are analyzed. As a caregiver exhibits signs of stress or potential burnout (e.g., frequent delays, low feedback scores, or reduced engagement levels), the system may trigger alerts or recommendations for intervention. These interventions may include workload adjustments, rest periods, or mental health support. Early detection and mitigation enhance caregiver well-being and maintain high-quality care delivery.

In some non-limiting embodiments or aspects, the system includes a performance optimization module that uses feedback-based machine learning models to refine or adapt care strategies. This module analyzes patient interactions, feedback, and caregiver actions to provide actionable recommendations. For example, if feedback indicates that a caregiver excels in certain types of care (e.g., post-surgical recovery), the system may suggest prioritizing similar care requests. Additionally, the model may identify areas for improvement, such as communication style or adherence to specific care protocols, and recommends targeted training or process adjustments. By continuously learning from real-time data, the system supports ongoing improvements in care quality and efficiency.

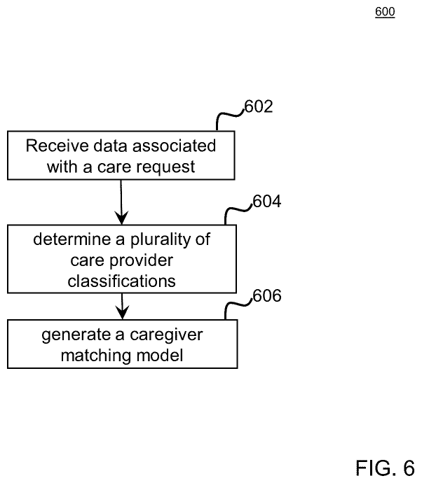

Referring now to FIG. 6, illustrated is a flowchart of a non-limiting aspect or embodiment of a process 600 for determining a care provider based on a care request. In some non-limiting embodiments or aspects, one or more of the functions described with respect to process 600 may be performed (e.g., completely, partially, etc.) by request processing server 104. In some non-limiting embodiments or aspects, one or more of the steps of process 600 may be performed (e.g., completely, partially, and/or the like) by another device or a group of devices separate from and/or including request processing server 104, such as user device 102 and/or one or more care provider devices 106a-106n.

As shown in FIG. 6, at step 602, process 600 may include receiving data associated with a care request. For example, request processing server 104 may collect care request data, including patient conditions, preferences, geographic location, or urgency levels, from user device 102. These input variables (e.g., medical condition, patient age, and priority levels) form part of a dataset used to optimize matching decisions.

As shown in FIG. 6, at step 604, process 600 includes determining a plurality of care provider classifications. Matching system 102 include machine learning models, trained using supervised learning methodologies, to classify care providers based on care provider attributes such as experience, specialization, and past performance. For example, care provider matching system 102 determines a plurality of care provider classifications based on care provider attributes associated with the plurality of care requests or a portion of the plurality of care requests, wherein a care provider classification identifies a type of care provided by a respective care provider. For example, care provider taxonomy may encompass classifications such as primary care, specialized care, pediatric care, and/or the like, covering each care type or a subset of care types based on geographic region, as detailed in Table 1 (above).

In some non-limiting embodiments or aspects, a care provider taxonomy for matching care providers includes categories to cover all care types, or alternatively, includes only a portion of care types, such as those active in a geographic region. Examples include specializations like geriatric care or mental health.

In some non-limiting embodiments or aspects, the system applies both supervised and unsupervised learning techniques to refine classification. For example, unsupervised clustering techniques (see [0095]) are used to group care providers based on latent performance metrics such as patient satisfaction and appointment completion rates, improving care matching accuracy.

As shown in FIG. 6, at step 606, process 600 includes generating a care provider matching model. For example, care provider matching system 102 may leverage supervised learning methodologies, such as polynomial regression and classification models, described in, to predict key metrics such as wait times and satisfaction probabilities. Care provider matching system 102 may generate a care provider matching model from the care request data to determine a care provider classification or to further provide an inference based on one or more care provider or recipient features. Classifier constructor 106 uses combinations of operational, embedded, and recipient features to train the model using neural networks, optimizing classification accuracy. Classifier constructor 106, for example, uses historical data combined with caregiver-specific features (e.g., caregiver stress levels, session checklists, and real-time patient feedback) to train the model iteratively, optimizing for prediction accuracy.

Request processing server 104 may determine one or more weighted scores based on data associated with ratings of one or more care providers. For example, user feedback and other historical data enhance model precision. Request processing server 104 may further determine one or more weighted scores based on real-time and historical feedback. For example, Bayesian methodologies ([0094]) and constrained factorial designs may be applied to tune the machine learning model, weighting caregiver attributes (e.g., specialization or location) based on their impact on predicted patient outcomes such as satisfaction or reduced hospital visits.

In some non-limiting embodiments or aspects, by employing machine learning methods such as Response Surface Methodology (RSM), steepest ascent path optimization, and ANOVA-based Goodness of Fit evaluations, the care provider matching model refines its predictions. For example, the model generates weighted scores for care provider matches, considering patient health data, caregiver stress indicators, and caregiving outcomes.

The care provider matching model uses this comprehensive dataset to accurately predict care provider classifications, providing valuable and precise inputs for matching care recipients with appropriate care providers.

Request processing server 104 may further generate care signatures and anomaly signals to detect mismatches or risks in care assignments. For example, request processing server 104 may detect deviations in patient satisfaction trends or caregiver burnout risk and provide anomaly signals to improve care assignment outcomes or recommend stress mitigation actions for caregivers. These signatures incorporate real-time metrics and historical datasets, improving the robustness of care provider recommendations and patient outcomes.

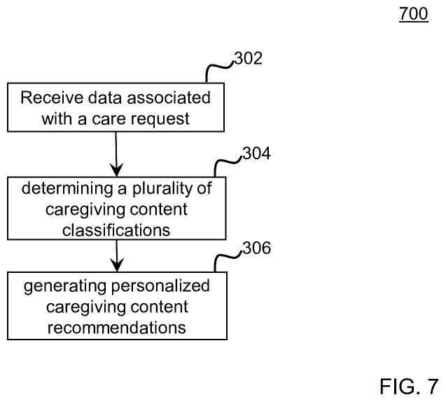

Referring now to FIG. 7, illustrated is a flowchart of a non-limiting aspect or embodiment of a process 700 for generating content and determining caregiving resources based on care requests and feedback. In some non-limiting embodiments or aspects, one or more of the functions described with respect to process 700 may be performed (e.g., completely, partially, etc.) by request processing server 104. In some non-limiting embodiments or aspects, one or more of the steps of process 700 may be performed (e.g., completely, partially, and/or the like) by another device or a group of devices separate from and/or including request processing server 104, such as user device 102 and/or one or more care provider devices 106a-106n.

As shown in FIG. 7, at step 702, process 700 may include receiving data associated with a care request. This data includes patient conditions, caregiver performance metrics, and feedback collected from user devices or care provider devices. The collected data is utilized for real-time content generation and resource optimization. The system aggregates caregiver performance metrics such as task completion rates, patient satisfaction scores, and stress levels. These metrics are compiled into performance summaries that highlight top-performing caregivers and areas requiring improvement. Summaries may provide actionable insights, enabling organizations to optimize caregiving strategies.

At step 704, process 700 includes determining a plurality of caregiving content classifications. For example, care provider matching system 102 uses machine learning models to classify caregiving content based on specific attributes (e.g., stress management, skill development). Dynamic taxonomies are developed to categorize caregiving tasks, such as “mobility assistance,” “wound care,” or “non-verbal communication.” These taxonomies provide a structured framework for organizing and labeling content.

In some non-limiting embodiments or aspects, the system continuously refines caregiving taxonomies by generating new caregiving task categories and subcategories in response to real-time feedback and caregiver needs. This allows the content to remain up-to-date and aligned with evolving caregiving challenges.

In some non-limiting embodiments or aspects, the system generates and associates seed word labels with caregiving tasks. These labels (e.g., “mobility,” “patient comfort”) guide the categorization and recommendation of relevant resources, ensuring that caregivers receive content specifically tailored to their tasks.

In addition to refining taxonomies, the system can dynamically translate caregiving content into different languages (e.g., from English to Chinese) and localize the content for specific regions or countries, such as the United States or China. For example, caregiver training materials may be adjusted to reflect cultural norms, healthcare policies, and language-specific nuances to ensure they are applicable and effective in the target region.

In some non-limiting embodiments or aspects, supervised learning techniques may be utilized to predict caregiving outcomes. These models leverage historical data, including caregiver stress levels, patient feedback, and care request urgency. Supervised learning algorithms, such as polynomial regression or decision trees, may predict key metrics, such as patient satisfaction or caregiver effectiveness. For instance, predicting the reduction in emergency visits or estimating caregiver stress levels based on past interactions can optimize care delivery.

Similarly, unsupervised learning techniques may identify hidden patterns in caregiver performance data. Clustering methods may group caregivers based on their performance metrics, while association rule learning discovers relationships between caregiving tasks and patient outcomes. For example, the system could prioritize a caregiver specializing in chronic condition management for patients with similar long-term illnesses, improving overall care matching.

At step 706, process 700 includes generating personalized caregiving content recommendations. For example, based on real-time feedback and performance data, the system generates instructional materials, stress management guides, and skill-building resources for caregivers. Classifier constructor 106 uses these data inputs to produce personalized recommendations aimed at improving caregiver performance and patient outcomes.

In some non-limiting embodiments or aspects, the system generates specific resources, such as guided exercises or tutorials tailored to real-time caregiver feedback. For example, if a caregiver is flagged for needing additional support in wound care, the system may provide a detailed tutorial on dressing changes or infection prevention. Personalized caregiving resources can also be adapted based on geographic or cultural factors. For instance, stress management strategies for caregivers in the United States may focus on leveraging local mental health resources, while those in China may highlight traditional practices such as Tai Chi or meditation. This ensures that recommendations are contextually relevant, increasing caregiver engagement and effectiveness.

In some non-limiting embodiments or aspects, the process 700 employs constrained factorial designs and response surface methodology (RSM) for iterative optimization of caregiving content. For example, supervised learning models may use constrained experimental designs to establish causality between caregiver actions and patient outcomes. By using techniques like ANOVA (Analysis of Variance) for goodness of fit, the system ensures that its predictions are both accurate and actionable. The entirety of the article titled “Experimental Designs When There Are One or More Factor Constraints” by George E. P. Box and Ian Hau, published in the Journal of Applied Statistics, Vol. 28, No. 8, November 2001, pages 973-989, is hereby incorporated by reference in its entirety for all purposes. This paper provides detailed methodologies and theoretical foundations for experimental designs under linear constraints, which are relevant to the present invention. Specifically, the methodologies described therein support the invention's approach to optimizing experimental setups for training machine learning models with constrained factors to enhance causal inference.

In some non-limiting embodiments or aspects, the system optimizes caregiver assignments based on dynamically updated scores to ensure the most effective allocation of resources. This includes considering patient needs, caregiver strengths, and real-time feedback to improve caregiving outcomes.

In some non-limiting embodiments or aspects, the system generates caregiver signatures and anomaly signals. For example, caregiver signatures incorporate aggregated performance data and real-time feedback, enabling the system to detect anomalies such as caregiver stress or mismatched assignments. These signatures enhance the robustness of content recommendations and resource allocation strategies.

Although the above methods, systems, and computer program products have been described in detail for the purpose of illustration based on what is currently considered to be the most practical and preferred embodiments or aspects, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, one or more features of any embodiment or aspect may be combined with one or more features of any other embodiment or aspect.

What is claimed is:

1. A computer-implemented method, comprising:
receiving data associated with a care request, the data associated with the care request transmitted by a device associated with a care recipient;
determining one or more values corresponding to one or more parameters of the care request;
determining one or more updated weighted scores corresponding to one or more care providers based on the one or more values corresponding to the one or more parameters of the care request, wherein the one or more weighted scores are determined by comparing values associated with one or more attributes of the care recipient to values associated with one or more attributes of the care providers, and incorporating updated data associated with at least one rating of at least one care provider;
selecting a care provider from among the one or more care providers based on the one or more updated weighted scores;
generating a care request for the selected care provider and transmitting the care request to a device associated with the selected care provider;
receiving updated data from at least one device associated with a user, the updated data regarding the care provided; and
dynamically updating the weighted scores for the care providers based on the updated data to improve the accuracy of future care provider selections.

2. The computer-implemented method of claim 1, wherein receiving the data associated with the care request comprises:
receiving data associated with one or more attributes of the care recipient, the one or more attributes of the care recipient comprising:
an identifier of the care recipient,
a rating of the care recipient,
a location associated with the care recipient,
a date for the care request,
the frequency of performance of a service involved in the care request,
a requested start time for the care request,
a requested end time for the care request,
an indication of whether the care request is a one-time care request or a repeating care request,
one or more services involved in the care request, and
one or more demographic requests associated with the care request.

3. The computer-implemented method of claim 1, further comprising:
receiving data associated with or more attributes of a care provider, the one or more attributes of the care provider comprising:
an identifier of the care provider,
a rating of the care provider;
a location associated with the care provider,
availability of the care provider,
clinical skills and competency of the care provider;
one or more times of one or more dates that the care provider is unavailable, demographics of the care provider,
a method of payment associated with the care request, and
qualifications of the care provider.

4. The computer-implemented method of claim 1, wherein determining the one or more weighted scores corresponding to one or more care providers comprises solving a constrained optimization problem,
wherein values associated with the one or more attributes of the care recipient and the care providers are subject to one or more linear constraints, ensuring compliance with predefined care requirements and resource limitations.

5. The computer-implemented method of claim 1, further comprising:
receiving data associated with one or more rules associated with one or more preferences of the care recipient.

6. The computer-implemented method of claim 1, wherein, receiving the data associated with the one or more rules associated with one or more preferences of the care recipient comprises:
transmitting a request to the device associated with the care recipient for input specifying the one or more rules associated with the one or more preferences of the care recipient; and
receiving the data associated with one or more rules associated with one or more preferences of the care recipient,
wherein, selecting the care provider from among the one or more care providers based on the one or more weighted scores comprises:
selecting the care provider from among the one or more care providers based on the one or more weighted scores and one or more rules associated with one or more preferences of the care recipient.

7. The computer-implemented method of claim 1, further comprising:
determining one or more rules associated with one or more preferences of the care recipient based on the one or more attributes of the care recipient,
wherein selecting the care provider from among the one or more care providers based on the one or more weighted scores comprises:
selecting the care provider from among the one or more care providers based on the one or more weighted scores and one or more rules associated with one or more preferences of the care recipient.

8. The computer-implemented method of claim 1, further comprising:
selecting data associated with one or more examples of media from a library of media, wherein the selection is based on the attributes of the care provider selected from among the one or more care providers and the attributes of the care request; and
transmitting the data associated with the one or more examples of media to a device associated with the care provider that was selected from among the one or more care providers, wherein the transmitted media includes instructional content, contextual information, or training material tailored to the care request.

9. The computer-implemented method of claim 1, wherein selecting the care provider from among the one or more care providers comprises:
formulating a constrained optimization model based on historical and real-time data;
applying linear constraints to the model, including, but not limited to, geographical proximity, availability windows, and competency requirements; and
solving the model to identify the care provider that optimally balances care recipient preferences and provider attributes while satisfying the linear constraints.

10. A care management system, comprising:
at least one processor configured to:
receive data associated with a care request, the data associated with the care request transmitted by a device associated with a care recipient;
determine one or more values corresponding to one or more parameters of the care request;
determine one or more updated weighted scores corresponding to one or more care providers based on the one or more values corresponding to the one or more parameters of the care request, wherein the one or more weighted scores are determined by comparing values associated with one or more attributes of the care recipient to values associated with one or more attributes of the care providers, and incorporating updated data associated with at least one rating of at least one care provider;
select a care provider from among the one or more care providers based on the one or more updated weighted scores;
generate a care request for the selected care provider and transmitting the care request to a device associated with the selected care provider;
receive updated data from at least one device associated with a user, the updated data regarding the care provided; and
dynamically update the weighted scores for the care providers based on the updated data to improve the accuracy of future care provider selections.

11. The care management system of claim 10, wherein the at least one processor is further configured to:
receive data associated with one or more attributes of the care recipient, the one or more attributes of the care recipient comprising at least one of:
an identifier of the care recipient,
a rating of the care recipient,
a location associated with the care recipient,
a date for the care request,
the frequency of performance of a service involved in the care request,
a requested start time for the care request,
a requested end time for the care request,
an indication of whether the care request is a one-time care request or a repeating care request,
one or more services involved in the care request, or
one or more demographic requests associated with the care request.

12. The care management system of claim 10, wherein the at least one processor is further configured to:
receive data associated with or more attributes of a care provider, the one or more attributes of the care provider comprising at least one of:
an identifier of the care provider,
a rating of the care provider;
a location associated with the care provider,
availability of the care provider,
clinical skills and competency of the care provider;
one or more times of one or more dates that the care provider is unavailable,
demographics of the care provider,
a method of payment associated with the care request, or
qualifications of the care provider.

13. The care management system of claim 10, wherein determining the one or more weighted scores corresponding to one or more care providers comprises solving a constrained optimization problem,
wherein values associated with the one or more attributes of the care recipient and the care providers are subject to one or more linear constraints, ensuring compliance with predefined care requirements and resource limitations.

14. The care management system of claim 10, wherein the at least one processor is further configured to:
receive data associated with one or more rules associated with one or more preferences of the care recipient.

15. The care management system of claim 10, wherein the at least one processor is further configured to:
transmit a request to the device associated with the care recipient for input specifying the one or more rules associated with the one or more preferences of the care recipient; and
receive the data associated with one or more rules associated with one or more preferences of the care recipient,
wherein, the at least one processor when selecting the care provider from among the one or more care providers based on the one or more weighted scores is further configured to:
select the care provider from among the one or more care providers based on the one or more weighted scores and one or more rules associated with one or more preferences of the care recipient.

16. The care management system of claim 10, wherein the at least one processor is further configured to:
determine one or more rules associated with one or more preferences of the care recipient based on the one or more attributes of the care recipient,
wherein the at least one processor when selecting the care provider from among the one or more care providers based on the one or more weighted scores is further configured to:
select the care provider from among the one or more care providers based on the one or more weighted scores and one or more rules associated with one or more preferences of the care recipient.

17. The care management system of claim 10, wherein the at least one processor is further configured to:
select data associated with one or more examples of media from a library of media, wherein the selection is based on the attributes of the care provider selected from among the one or more care providers and the attributes of the care request; and
transmit the data associated with the one or more examples of media to a device associated with the care provider that was selected from among the one or more care providers, wherein the transmitted media includes instructional content, contextual information, or training material tailored to the care request.

18. The care management system of claim 10, wherein the at least one processor when selecting the care provider from among the one or more care providers is further configured to:
formulate a constrained optimization model based on historical and real-time data;
apply linear constraints to the model, including, but not limited to, geographical proximity, availability windows, and competency requirements; and
solve the model to identify the care provider that optimally balances care recipient preferences and provider attributes while satisfying the linear constraints.

19. A computer program product comprising at least one non-transitory computer-readable medium including one or more instructions that, when executed by at least one processor, cause the at least one processor to:
receive data associated with a care request, the data associated with the care request transmitted by a device associated with a care recipient;
determine one or more values corresponding to one or more parameters of the care request;
determine one or more updated weighted scores corresponding to one or more care providers based on the one or more values corresponding to the one or more parameters of the care request, wherein the one or more weighted scores are determined by comparing values associated with one or more attributes of the care recipient to values associated with one or more attributes of the care providers, and incorporating updated data associated with at least one rating of at least one care provider;
select a care provider from among the one or more care providers based on the one or more updated weighted scores;
generate a care request for the selected care provider and transmitting the care request to a device associated with the selected care provider;
receive updated data from at least one device associated with a user, the updated data regarding the care provided; and
dynamically update the weighted scores for the care providers based on the updated data to improve the accuracy of future care provider selections.

20. The computer program product of claim 19, comprising at least one non-transitory computer-readable medium including one or more instructions that, when executed by at least one processor, cause the at least one processor to:
receive data associated with one or more rules associated with one or more preferences of the care recipient,
wherein determining the one or more weighted scores corresponding to one or more care providers comprises solving a constrained optimization problem, and
wherein values associated with the one or more attributes of the care recipient and the care providers are subject to one or more linear constraints, ensuring compliance with predefined care requirements and resource limitations.
